(19) United States
(12) Patent Application Publication
(10) Pub. No.: US 2008/0167493 A1
(43) Pub. Date: Jul. 10, 2008

(54) METHODS FOR CONVERSION OF TYROSINE TO P-HYDROXYSTYRENE AND P-HYDROXYCINNAMIC ACID

(76) Inventors: STEVEN W. SHUEY, Landenberg, PA (US); Mukesh C. Shah, Hockessin, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 11/961,072

(22) Filed: Dec. 20, 2007

Related U.S. Application Data
(60) Provisional application No. 60/883,359, filed on Jan. 4, 2007.

(51) Int. Cl. C07C 51/347

(52) U.S. Cl. 562/471

(57) ABSTRACT

Three different reaction steps were combined to provide methods for preparing p-hydroxystyrene and p-hydroxycinnamic acid monomers from tyrosine. The three steps include reductive alkylation of tyrosine, followed by oxidation to the N-oxide, and thermal Cope elimination. During Cope elimination, either p-hydroxycinnamic acid or p-hydroxystyrene was produced depending on the absence or presence of base, respectively. Additionally, p-acetoxystyrene may be prepared by reacting the prepared p-hydroxystyrene either directly or after isolation with an acetylating agent.

This application claims the benefit of U.S. Provisional Application, 60/883359, filed Jan. 4, 2007.

### FIELD OF INVENTION

The invention relates to the field of organic synthesis. More specifically, the invention relates to methods for preparing p-hydroxystyrene and p-hydroxycinnamic acid monomers from tyrosine in three steps including reductive alkylation of tyrosine, followed by oxidation to the N-oxide, and thermal Cope elimination.

### BACKGROUND OF THE INVENTION

Hydroxystyrenes, such as p-hydroxystyrene (pHS, also called HSM; also known as p-vinylphenol) and acetylated derivatives thereof, such as p-acetoxystyrene (pAS, also called ASM), are aromatic compounds that have potential utility in a wide variety of industrial applications. For example, these compounds have applications as monomers for the production of resins, elastomers, adhesives, coatings, automotive finishes, inks and photoresists, as well as in electronic materials. They may also be used as additives in elastomer and resin formulations. The related compound p-hydroxycinnamic acid (pHCA) is a useful monomer for the production of Liquid Crystal Polymers (LCP). LCPs are used in liquid crystal displays, and in high speed connectors and flexible circuits for electronic, telecommunication, and aerospace applications. Because of their resistance to sterilizing radiation and their high oxygen and water vapor barrier properties, LCPs are used in medical devices, and in chemical and food packaging. In addition, pHCA is useful in the preparation of sunscreen compounds.

A number of methods for the chemical synthesis of hydroxystyrenes and acetylated derivatives thereof are known. However, these methods generally require expensive reagents, harsh conditions, and give relatively low yields. Examples of starting reagents used in the chemical synthesis of pHS include p-hydroxycinnamic acid (pHCA; Sovish J. Org. Chem. 24:1345-1347 (1959); U.S. Pat. No. 5,274,060), p-hydroxybenzaldehyde (U.S. Pat. No. 4,316,995), ortho or para-hydroxyarylcarboxylic acids (Australian Patent Application No. 7247129), caffeic acid (U.S. Pat. No. 5,324,804), trans-3,5-di-tert-butyl-4-hydroxycinnamic acid (Munteanu et al. J. Thermal Anal. 37:411-426 (1991)), p-alpha-amino-ethylphenol (U.S. Pat. No. 5,493,062), and hydroxybenzaldehydes and malonic acid (Simpson et al., Tetraheron Lett. 46: 6893 (2005)).

Chemical synthesis of pHCA is known (see for example JP 2004231541 and JP 200414943). Additionally pHCA, an intermediate in the lignin biosynthetic pathway is commonly extracted from plant tissue [Plant Biochemistry, Ed. P. M. Dey, Academic Press, (1997)] and methods of its isolation and purification are known [R. Benrief, et al., Phytochemistry, 47, 825-832; WO 972134; Bartolome et al., Journal of the Science of Food and Agriculture (1999), 79(3), 435-439. These methods are time consuming, expensive and cumbersome. A more facile method of production is therefore needed for the inexpensive and large scale synthesis of this monomer.

Tyrosine, (S)-2-amino-3-(4-hydroxy-phenyl)propanoic acid, provides a readily available and relatively low cost reagent which has potential of being a reagent for producing pHS and pHCA. Tyrosine was used as the starting reagent in the synthesis of (S)-4-(2-chloro-3-(4-n-dodecyloxy)phenylpropionato)-4′4(2-methyl)butyloxy-biphenylcarboxylate (CDPMBB) in Kumar and Pisipati (Z. Naturforsch. 57a:803-806 (2002)). The initial reaction was diazochlorination of tyrosine by nucleophilic substitution in the presence of sodium nitrite to form (S)-2-chloro-3-(4-hydroxy)phenyl propionic acid. Yields of the reaction were poor, and by-products resulting from nitration or chlorination of the aromatic ring made purification difficult.

Souers et al., (Synthesis 4:583-585 (1999)) teach the bromination of side chain protected tyrosine and tyrosine t-butyl ether in the presence of HBr, KBr, and sodium nitrite. The presence of the protecting group on the phenol moiety complicates the use of this product as a reagent for producing pHS and pHCA.

Co-owned and co-pending U.S. patent application Ser. No. 11/369422 discloses a method for synthesis of pHS and pAS from tyrosine using HBr and NaNO₂ to form an isomeric mixture of brominated tyrosine intermediates which are subsequently converted to p-hydroxystyrene in the presence of a base catalyst.

Elimination of the amine function of amino acids to give olefins has been accomplished by the Hofmann elimination. However, for tyrosine, during the quaternization of the amine, the phenol also gets methylated, thus pHCA is not obtainable. (Ulrich (1949) Helv. Chim. Acta 32, 681-686).

Dialkyl amino acids have been prepared by reaction of sodium triacetoxyborohydride and free amino acids with various aldehydes (Levadala et al. (2004) Synthesis 11:1759-1766). This and other related methods use costly reagents and/or require multiple steps. Alkylation with alkylating reagents, such as methyl iodide of dimethylsulfate, have been used for alkylation of amino acids but require multiple steps, give very low yields and it is very difficult to achieve dimethylation (Ulrich (1949) Helv. Chim. Acta 32, 681-686).

Reduction using metal catalyzed dehydrogenation has been described (Song et al. (2000) Tetrahedron Lett 41: 8225-8230). Oxidation of a tertiary amine to an amine oxide has been accomplished using oxidants (Ikutani (1968) Bull. Chem. Soc. Jpn. 41:1679-1681).

A combination of dialkylation and dehydrogenation reactions with additional step(s) to create a commercially viable method for synthesis of pHCA and pHS has not been described. In particular, elimination of amine oxides from amino acids to give olefins, specifically using tyrosine derivatives as substrates, is a missing component of a process for synthesis of pHCA and pHS.

There is a need for a method for the chemical synthesis of pHS and pHCA from tyrosine which is efficient, that produces product in high yield, that avoids the complications of by-products generated from the use of a side chain protected tyrosine, and that is environmentally friendly. Applicants have solved the stated problem by the discovery of new methods for conversion of tyrosine to pHS and pHCA that meet these criteria.

### SUMMARY OF THE INVENTION

Methods are provided for the synthesis of p-hydroxycinnamic acid (pHCA), p-hydroxystyrene (pHS) and p-acetoxystyrene (pAS). In a first step, tyrosine undergoes reductive alkylation to produce N,N dialkyltyrosine as a first intermediate. In the second step, N,N dialkyltyrosine is oxidized to form N,N dialkyltyrosine N-oxide as a second intermediate. In a third step reaction either pHCA or pHS is produced in the absence or presence of base, respectively, by thermal Cope elimination. Additionally, pHS produced by this method may be converted directly or after isolation to pAS in the presence of an acetylating agent.

Accordingly, in one embodiment the invention provides a method for the synthesis of p-hydroxycinnamic acid comprising:
a) reacting tyrosine over a metal hydrogenation catalyst with a reaction mixture comprising:
   i) at least two equivalents of an aldehyde; and
   ii) hydrogen or a hydrogen donor;
to form an N,N-dialkyltyrosine product having the general structure of Formula l:

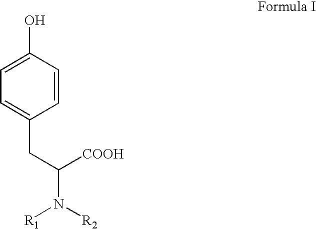

wherein R₁ and R₂ are C1 to C10 linear or branched alkyls;
b) optionally isolating the N,N-dialkyltyrosine product;
c) reacting the N,N dialkyltyrosine product with an oxidizing agent to form N,N dialkyltyrosine N-oxide having the general structure of Formula II:

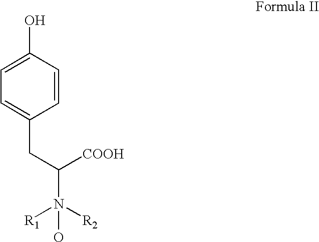

wherein R₁ and R₂ are C1 to C10 linear or branched alkyls;
d) optionally isolating the N,N dialkyltyrosine N-oxide product;
e) heating the N,N dialkyltyrosine N-oxide product to form a mixture of dialkylhydroxylamine by-product and p-hydroxycinnamic acid; and
f) removing dialkylhydroxylamine by-product of step (e) wherein the p-hydroxycinnamic acid product is stabilized.
   In another embodiment the invention provides a method for the synthesis of p-hydroxystyrene comprising:
a) reacting tyrosine over a metal hydrogenation catalyst with a reaction mixture comprising:
   i) at least two equivalents of an aldehyde; and
   ii) hydrogen or a hydrogen donor;
to form an N,N-dialkyltyrosine product having the general structure of Formula I:

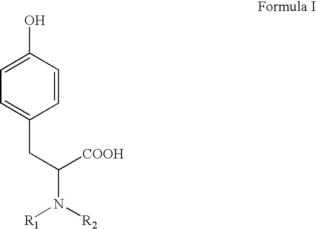

wherein R₁ and R₂ are C1 to C10 linear or branched alkyls;
b) optionally isolating the N,N-dialkyltyrosine product;
c) reacting the N,N dialkyltyrosine product with an oxidizing agent for form an N,N dialkyltyrosine N-oxide product having the structure of Formula II:

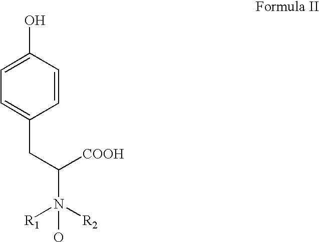

wherein R₁ and R₂ are C1 to C10 linear or branched alkyls;
d) optionally isolating the N,N dialkyltyrosine N-oxide product; and
e) heating the N,N dialkyltyrosine N-oxide product in the presence of a base wherein thermal decomposition occurs to form p-hydroxytsyrene.
   In another embodiment the invention provides a method for the synthesis of p-acetoxystyrene comprising:
a) reacting tyrosine over a metal hydrogenation catalyst with a reaction mixture comprising:
   i) at least two equivalents of an aldehyde; and
   ii) hydrogen or a hydrogen donor;
to form an N,N-dialkyltyrosine product having the general structure of

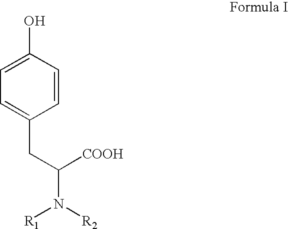

wherein R₁ and R₂ are C1 to C10 linear or branched alkyls;
b) optionally isolating the N,N-dialkyltyrosine product;
c) reacting the N,N dialkyltyrosine product with an oxidizing agent to form an N,N dialkyltyrosine N-oxide product having the general structure of Formula II:

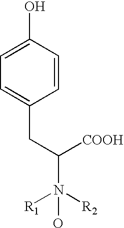

wherein R₁ and R₂ are C1 to C10 linear or branched alkyls;
d) optionally isolating the N,N dialkyltyrosine N-oxide product;
e) heating the N,N dialkyltyrosine N-oxide product with in the presence of base wherein thermal decomposition occurs to produce p-hydroxystyrene;
f) optionally isolating the p-hydroxystyrene of (e); and
g) reacting the p-hydroxystyrene with an acetylating agent wherein p-acetoxystyrene is formed.

### DETAILED DESCRIPTION OF THE INVENTION

The invention provides methods for preparing p-hydroxystyrene (pHS) and p-hydroxycinnamic acid (pHCA) from tyrosine that are high yielding and environmentally friendly. The methods of synthesis includes three steps. The first step reaction is a dialkylation reaction of the free amino acid tyrosine where the nitrogen of tyrosine is alkylated to produce N,N dialkyltyrosine. The tertiary amine of this first intermediate is then oxidized to an amine oxide forming N,N dialkyltyrosine N-oxide in a second step reaction. This second intermediate then is subjected to a Cope reaction in a third step, where there is thermal decomposition of the amine oxide, forming an alkene. In the absence of added base and with removal of dialkylhydroxylamine by-product, (Reaction IIIA below), pHCA is the product of the Cope reaction. In the presence of base, which can be the dialkylhydroxylamine by-product alone, an added base, or both the dialkylhydoxylamine and an added base (Reaction IIIB below), pHS is the product of the Cope reaction.

In an additional embodiment, the pHS produced in Reaction IIIB is converted to p-acetoxystyrene (pAS) in the presence of an acetylating agent.

Both pHS and pAS find utility as monomers for use in commercial resins, elastomers, adhesives, coatings, automotive finishes, inks, photoresists, electronic materials, and additives in elastomer and resin formulations. pHCA is a useful monomer for production of Liquid Crystal Polymers (LCP). LCPs are used in liquid crystal displays, and in high speed connectors and flexible circuits for electronic, telecommunication, and aerospace applications, as well as in medical devices, and in chemical and food packaging.

The following definitions are used herein and should be referred to for interpretation of the claims and the specification.

“p” means para.

“pAS” is the abbreviation used for para-acetoxystyrene which is also represented as p-acetoxystyrene or 4-acetoxystyrene.

“pHS ” is the abbreviation used for para-hydroxystyrene which is also represented as p-hydroxystyrene or 4-hydroxystyrene.

“pHCA” is the abbreviation used for para-hydroxycinnamic acid which is also represented as p-hydroxycinnamic acid or 4-hydroxycinnamic acid.

The term “yield” as used herein refers to the amount of product produced in a chemical reaction. The yield is typically expressed as a percentage of the theoretical yield for the reaction. The term “theoretical yield” means the predicted amount of product to be expected based on the amount of substrate initially present and the stoichiometry of the reaction.

The term “polar” as applied to solvents of the invention refers to solvents characterized by molecules having sizable permanent dipole moments.

The term “aprotic” as applied to the solvents of the invention refers to a solvent that is incapable of acting as a labile proton donor or acceptor.

The term “protic” as applied to the solvents of the invention refers to a solvent that is capable of acting as a labile proton donor or acceptor.

The term “polar organic solvent mixture” refers to a mixture of organic solvents comprising at least one polar solvent.

The term “aprotic, polar organic solvent mixture” refers to a mixture of organic solvents comprising at least one aprotic, polar solvent.

The term “complete” as it is used relative to the term of a chemical reaction refers to the point where the maximum product has been formed under the conditions of the reaction.

All ranges given herein include the end of the ranges and also all the intermediate range points.

The present invention provides methods for the production of pHCA and pHS from unprotected tyrosine in three steps. In a first embodiment, base is eliminated in the third step to allow accumulation of pHCA. In a second embodiment, base is included in the third step to favor pHS production and accumulation. In a third embodiment, pHS is produced as in the second embodiment, then acetylated to form pAS.

### First Step Reaction

The first step of the present method proceeds according to the following Reaction I:

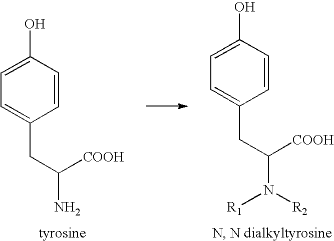

wherein R₁ and R₂ are C1 to C10 linear or branched alkyls. For example, in this reaction unprotected tyrosine may be dimethylated to produce N,N dimethyltyrosine.

Tyrosine substrate may be derived from any source. Tyrosine may be chemically synthesized or biologically produced, such as through fermentation as described in commonly owned and co-pending, US 20050148054A1, which is herein incorporated by reference. Tyrosine is commercially available, for example, from Aldrich (Milwaukee, Wis.). D-tyrosine, L-tyrosine, or a mixture of D- and L-tyrosine may be used.

Dialkylation of tyrosine is accomplished by metal catalyzed alkylation, an example of which is described in Song et al. ((2000) Tetrahedron Lett 41: 8225-8230). Metal hydrogenation catalysts that may be used include palladium on carbon, palladium salts on carbon such as palladium hydroxide or palladium acetate on carbon, other supported types of palladium such as silk-palladium catalyst (described in Ikutani, (1968) Bull. Chem. Soc. Jpn, 41,1679-1681), Raney nickel, platinum, ruthenium, rhodium and other metal hydrogenation catalysts that are well known to one skilled in the art. Particularly suitable catalysts are palladium on carbon, pallaidium hydroxide on carbon (Pd(OH)₂/C or Pd/C) and Raney nickel. Typically the metal catalyst is present in the reaction at a concentration of about 1 mole % to about 50 mole % with respect to the substrate tyrosine. The concentration of catalyst will also be varied depending on the concentration of the other reactants as is commonly known to those of skill in the art.

Hydrogen gas or a hydrogen donor is included in the reaction with the metal hydrogenation catalyst. A hydrogen donor is a molecule that will undergo reaction such that two hydrogen atoms are donated and become bonded to the metal. Examples of hydrogen donors are cylohexene, cyclohexadiene, limonene and ammonium formate.

The first step reaction includes at least two equivalents of an aldehyde. Examples of aldehydes that may be used include formaldehyde, benzaldehyde, and propionaldehyde. Particularly suitable is the use of formaldehyde, producing N,N dimethyltyrosine as the reaction product.

A wide variety of solvents may be used in the first step reaction, including water, HCl, alcohols such as methanol, ethanol or isopropanol, polar aprotic solvents such as DMF, DMAc, and THF, and solvent mixtures of any of these with water. A particularly suitable solvent for the first reaction is water.

The order of addition of the reactants is not critical. For example, tyrosine, formaldehyde, Pd/C and solvent are combined in any order, and put under hydrogen. However, for safety and practicality reasons the catalyst is usually added first under an inert atmosphere, followed by the other reagents and solvent. In addition to hydrogen providing a reducing agent, hydrogen pressure may be used to increase the reaction rate. The pressure applied is typically between about 15 psi and 5000 psi. Agitation may also be used to increase the reaction rate.

The reaction may be run at a temperature that is between about 20° C. and about 85° C., and preferably between about 25° C. and about 75° C. The reaction times may vary depending on conditions and reactant concentrations, however most reactions will be complete in between about 1 and 8 hours. Monitoring for completion of the reaction is known to one skilled in the art, and can be performed using a method such as HPLC analysis, for example.

Following the reaction, the product of the reaction may be purified using typical methods that may include steps such as filtration, concentration, precipitation, chromatography and/or crystallization. The pH may be adjusted for different purification steps. For example, for filtration the pH is adjusted so that all components are in solution. A pH of about 6 is typically used when filtering the dimethyltyrosine product. For crystallization, the pH may be adjusted for optimizing crystallization, such as using pH of about 7 for the dimethyltyrosine product.

As is typical of reactions, the yields of N,N dialkyltyrosine will vary depending on the relative concentrations of reactants and the temperature and pressure of the reaction. One of skill in the art will readily be able to determine the preferred concentration of reactants for maximum yield of product. Some non-limiting variations are illustrated in Table 2 of the Examples. Reagent concentrations tested that gave the highest yields include: 1) 10 mol % of Pd/C, 1 M concentration of tyrosine, with water as solvent, 500 psi hydrogen, at 25° C. for 1 hour or at 75° C. for 8 hours; and 2) 10 mol % of Pd/C, 0.1 M concentration of tyrosine, with water as solvent, 15 psi hydrogen, at 75° C. for 8 hours.

### Second Step Reaction

In the present method, the N,N dialkyltyrosine intermediate is oxidized in a second step reaction to form N,N dialkyltyrosine N-oxide according to the following Reaction II:

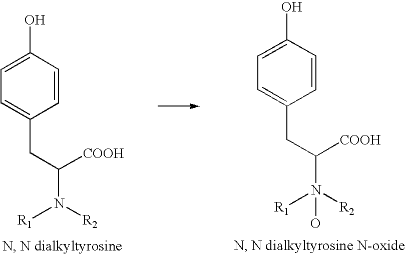

wherein R₁ and R₂ are C1 to C10 linear or branched alkyls. For example, in this reaction N,N dimethyltyrosine is oxidized to produce N,N dimethyltyrosine N-oxide.

The second step reaction may be accomplished as described by Ikutani ((1968) Bull. Chem. Soc. Jpn. 41:1679-1681) for the oxidation of a tertiary amine. The second step reaction proceeds in the presence of an oxidizing agent. Oxidants such as meta chloroperbenzoic acid (mCPBA), peroxides or hydroperoxides may be used. A particularly suitable oxidant is hydrogen peroxide. The solvent for the second step reaction may be an acidic, neutral, or basic aqueous medium. Particularly suitable is an acidic aqueous solvent.

The reaction may be run at a temperature that is between about 0° C. and about 150° C., and preferably between about 40° C. and about 70° C. The reaction times will vary depending on conditions and reactant concentrations, however most reactions will be complete in between about 1 and about 10 hours.

The N,N dialkyltyrosine N-oxide product may be purified. Prior to purification, the oxidant is removed by adding a reductant, distillation, adding a decomposition enzyme such as peroxidase for peroxides, or other suitable method. For example, when using hydrogen peroxide as the oxidant, following the reaction the hydrogen peroxide may be removed by repeated distillation. Water is added and the sample is distilled to about half volume. This procedure is repeated until the distillate gives a negative reaction to Kl/starch paper.

The product of the reaction is purified using typical methods of drying, crystallization, filtration, chromatography, and/or washing.

### Third Step Reaction

Applicants have found that heating the N,N dialkyltyrosine N-oxide allows this compound to undergo a Cope reaction (Cope and LeBel (1960) J. Am. Chem. Soc. 82:4656-4662) to produce pHCA or pHS, depending on the conditions used. In one embodiment, base is removed from the reaction as it is formed and the thermal decomposition that eliminates the amine oxide produces p-hydroxycinnamic acid (pHCA) according to the following Reaction IIIA.

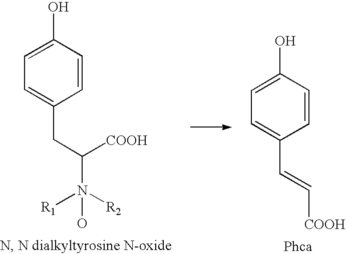

wherein R₁ and R₂ are C1 to C10 linear or branched alkyls.

During thermal decomposition of the amine oxide, dialkylhydroxylamine is formed as a by-product. To maximize production of pHCA, this base that is formed in the reaction is removed by distillation, so that base is not retained in the reaction mixture. In the presence of dialkylhydroxylamine, or other base, pHCA may be converted to pHS, thereby reducing the yield of pHCA. Removing the dialkylhydroxylamine stabilizes the pHCA product.

A variety of solvents may be used in the third step IIIA reaction. Examples of solvents include THF and other ethers, glyme diglyme, toluene, and polar aprotic solvents such as DMAc and DMF. DMAc and DMF are particularly suitable.

The IIIA reaction may be run at a temperature that is between about 60° C. and about 150° C., and preferably between about 110° C. and about 130° C. Reaction times to completion will vary, however, reaction times of about 15 minutes to about 2 hours may be used. Most suitable are reaction times of between about 15 minutes and about 45 minutes.

In a second embodiment of the third step reaction, base is present in the reaction. In the presence of base, N,N dialkyltyrosine N-oxide is directly converted to pHS according to the following Reaction IIIB.

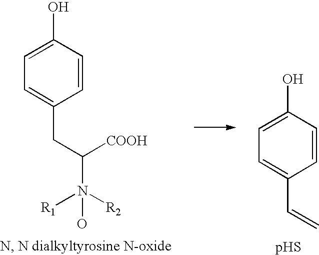

wherein R₁ and R₂ are independently selected from alkyls.

For production of pHS, dialkylhydroxylamine need not be removed, although it is still often desirable to remove this by-product as it is formed in the reaction as described in the IIIA reaction. For example, the dialkylhydroxylamine is typically removed when the pHS is subsequently acetylated as described below, polymerized, or otherwise modified. When the dialkylhydroxylamine is removed, additional base is included in the reaction. Alternatively, the dialkylhydroxylamine by-product may provide the base in the reaction to produce pHS. Additional base may be added to increase the rate of decarboxylation and the yield of pHS. The additional base, used with or without removal of dialkylhydroxylamine, may be any basic catalyst that is capable of facilitating the reaction to pHS. Such catalysts include, but are not limited to, potassium acetate, potassium carbonate, potassium hydroxide, sodium acetate, sodium carbonate, sodium bicarbonate, sodium hydroxide, magnesium oxide, pyridine, triethylamine, and other tertiary amines. Non-amine, basic catalysts are more suitable, including potassium carbonate, sodium carbonate, sodium hydroxide, and potassium acetate. Particularly suitable are weakly basic catalysts such as potassium acetate, potassium carbonate and sodium carbonate. Basic catalysts suitable in the present method are available commercially from, for example, EM Science (Gibbstown, N.J.) or Aldrich (Milwaukee, Wis.) and are present in the reaction in catalytic amounts.

The optimum concentration of basic catalyst will vary depending on the concentration of substrate, nature of the solvent used and reaction conditions. Typically, concentrations of about 1 mol % to about 30 mol % relative to the substrate, are used in the reaction mixture.

Solvents and temperatures for the Reaction IIIB are as described for Reaction IIIA. Reaction times are typically between about 30 minutes and about 10 hours. Longer times within this range are used when the dialkylhydroxylamine by-product is the only base present, while shorter times are sufficient when additional base is included in the reaction.

### Polymerization Inhibitors

Polymerization inhibitors may be added in the Reaction IIIB that produces pHS. Any suitable polymerization inhibitor that is tolerant of the temperatures required for the decarboxylation (second step) reaction as described in the invention may be used. Examples of suitable polymerization inhibitors include, but are not limited to, hydroquinone, hydroquinone monomethylether, 4-tert-butyl catechol, phenothiazine, N-oxyl (nitroxide) inhibitors, including Prostab® 5415 (bis(1-oxyl-2,2,6,6-tetramethylpiperidine-4-yl)sebacate, CAS#2516-92-9, available from Ciba Specialty Chemicals, Tarrytown, N.Y.), 4-hydroxy-TEMPO (4-hydroxy-2,2,6,6-tetramethylpiperidin-1-yloxy, CAS#2226-96-2, available from TCI America) and Uvinul® 4040 P (1,6-hexamethylene-bis(N-formyl-N-(1-oxyl-2,2,6,6-tetramethylpiperidine-4-yl)amine, available from BASF Corp., Worcester, Mass.).

### Polymerization Retarders

In some instances it may be advantageous to use a polymerization retarder in the Reaction IIIB. Polymerization retarders are well known in the art and are compounds that slow down the polymerization reaction but cannot prevent it altogether. Common retarders are aromatic nitro compounds such as dinitro-ortho-cresol (DNOC) and dinitrobutylphenol (DNBP). Methods for the preparation of polymerization retarders are common and well known in the art (see for example U.S. Pat. No. 6,339,177; Park et al., Polymer (Korea) (1988), 12(8), 710-19) and their use in the control of styrene polymerization is well documented (see for example Bushby et al., Polymer (1998), 39(22), 5567-5571).

Acetylation of p-Hydroxystyrene

In another embodiment of the invention pHS may be acetylated to p-acetoxystyrene (pAS) as shown in Reaction IV:

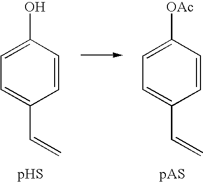

The pHS product may be transferred from the third step reaction to another vessel for acetylation, or the pHS product may be converted to an acetylated derivative by adding an acetylating agent directly to the reaction mixture after completion of the decarboxylation reaction.

For the acetylation process, organic solvents used should have the net characteristics of being both aprotic and polar. Thus direct acetylation of pHS in the Reaction IIIB mixture is effective when an aprotic, polar solvent is used in Reaction IIIB. A single aprotic, polar solvent may be used, or a mixture of aprotic, polar solvents may be used. Alternatively, an aprotic, polar solvent may be used in combination with a non-polar solvent; however, protic solvents are undesirable because they tend to consume acetylating agent due to their reactivity. Solvents suitable in the acetylation process include, but are not limited to, N,N-dimethylformamide, 1-methyl-2-pyrrolidinone, N,N-dimethylacetamide, dimethylsulfoxide, hexamethylphosphoramide, THF, and hexamethylphosphorous triamide. Particularly suitable solvents are N,N-dimethylformamide and N,N-dimethylacetamide.

Typically the acetylating agent is used in excess where a concentration of at least 1 mole equivalent as compared to the substrate is particularly suitable. Suitable acetylating agents include, but are not limited to, acetic anhydride and acetyl chloride.

The acetylation reaction may be carried out with high yield at temperatures ranging from about 0° C. to about 150° C., and more suitably at temperatures ranging from about 50° C. to about 140° C. One skilled in the art will recognize that a temperature at which both the substrate and the catalyst are soluble is preferred. The simplest approach is to add the acetylating agent just after completion of the amine oxide elimination reaction step and to perform the acetylation at the same temperature as the amine oxide elimination reaction. In one embodiment this is accomplished using DMAc or DMF in the third step reaction at a temperature that is between about 110° C. and about 130° C., then adding acetic anhydride for acetylation at the same temperature.

Isolation of pHCA, pHS and pAS Products

The pHCA product, pHS product or the pAS product may be isolated using any suitable method known in the art. For example, the solvent may be removed by reduced pressure distillation. The products may be further purified using vacuum distillation, recrystallization and/or chromatographic techniques that are well known in the art.

The resultant pHS or pAS may then be used as monomers for the production of, for example, resins, elastomers, adhesives, coatings, automotive finishes, inks, photoresists and as additives in elastomer and resin formulations. The pHCA may be used Liquid Crystal Polymers (LCP), which are used in liquid crystal displays, in high speed connectors and flexible circuits for electronic, telecommunication, and aerospace applications, as well as in medical devices and in chemical and food packaging.

### EXAMPLES

The present invention is further defined in the following Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### General Methods

The meaning of abbreviations used is as follows: “min” means minute(s), “h” means hour(s), “sec” means second(s), “mL” means milliliter(s), “L” means liter(s), “pL” means microliter(s), “μm” means micrometer(s), “mol” means mole(s), “mmol” means millimole(s), “g” means gram(s), “mg” means milligram(s), “M” means molar concentration, “m” means molal concentration, “eq” means equivalents, “v/v” means volume to volume ratio, “Pa” means pascal, “mPa” means millipascal, “psig” means pounds per square inch gauge, “HPLC” means high performance liquid chromatography, “DMF” means N,N-dimethylformamide, “DMAc” means N,N-dimethylacetamide, “NMP” means 1-methyl-2-pyrrolidinone, and “kPa” means kilopascal(s). “THF” is tetrahydrofuran

### Reagents:

All solvents were reagent grade and were obtained from Aldrich (Milwaukee, Wis.) unless noted otherwise. The basic catalysts used were obtained from Aldrich or EM Science (Gibbstown, N.J.).

### HPLC Methods:

The Agilent 1100 HPLC system was used with a reverse-phase Zorbax SB-C18 column (4.6 mm×150 mm, 3.5 μm, supplied by Agilent Technologies). The HPLC separation was achieved using a gradient combining two solvents: Solvent A, 0.1% trifluoroacetic acid in HPLC grade water and Solvent B, 0.1% trifluoroacetic acid in acetonitrile. The mobile phase flow rate was 1.0 mL/min. The solvent gradient used is given in Table 1. A temperature of 40° C. and a sample injection of 1 μL were used.

TABLE 1
Solvent Gradient Used for HPLC

| Time (min) | Solvent A | Solvent B |
|---|---|---|
| 0 | 95% | 5% |
| 10 | 100% | 0% |
| 12 | 100% | 0% |
| 12.5 | 95% | 5% |

Suitable calibration curves were generated as described above and used to determine wt % of pHS in each sample from HPLC peak areas. With this information and the total weight of the reaction mixture at each time point, the weight and moles of pHS versus time were calculated.

### Example 1

### Preparation of N,N-Dimethyltyrosine

Into a 200 mL stainless steel pressure vessel was added 25 g (0.138 mol) L-tyrosine (Aldrich), 24.64 g (0.304 mol) of a 37% solution of formaldehyde (Aldrich), 22.0 g of 15% Pd/C (Aldrich) and 66 mL water. The vessel was evacuated and then put under 500 PSI hydrogen and heated to 75° C. for 8 h. After cooling to room temperature, the contents of the vessel were transferred to an ehrlenmeyer flask and the pH of the solution was adjusted to 6 by addition of 1N HCl. The reaction mixture was filtered through a course sintered funnel packed with Celite 545 (EMD). The filtrate was then adjusted to pH 7 by addition of 1N sodium hydroxide. The clear filtrate was transferred to a round bottom flask and concentrated on a rotary evaporator at 40° C. until solids just started to form. Water was added to redissolve the solids and then 75 mL acetone was added. The solution was chilled in the refrigerator overnight, which gave white crystals. The solution was allowed to come to room temperature, filtered through paper, and the crystals washed with cold acetone. The mother liquor was concentrated and the procedure repeated to give a second crop of white crystals. The combined solids were dried in a vacuum oven at 85° C. overnight to give 21.23 g of the desired compound. (73.72% yield). The product was identified as N,N-dimethltyrosine by NMR analysis. ¹H NMR (DMSO-d6) δ 2.4(s,6H); δ2.7(m,1 H); δ2.9(m,1 H); δ3.3(m,1H); δ6.6(d,2H); δ7(d,2H). Analytical Calculated for C11H15NO3: C,63.14; H 7.23; N 6.69; O 22.94. Found C 62.29; H 7.3, N 6.62

### Example 2

### Preparation of N,N-Dimethyltyrosine N-Oxide

A solution of 5.0 gm of N,N-dimethyltyrosine (prepared as described in Example 1), 50 mL of glacial acetic acid and 25 mL 30 wt % hydrogen peroxide was warmed at 60° C. for 4.5 h. To the reaction mixture was added 50 mL of water, after which the mixture was concentrated to half volume under reduced pressure, keeping the temperature below 40° C. This procedure was repeated until the distillate gave a negative reaction to Kl/starch paper. To the resulting slurry was added 50 ml of ethanol to form a solution. The solution was dried under reduced pressure, and the procedure was repeated two times to give the crude product as a solid. The crude was recrystallized from water/acetone. The solid product was filtered, washed with cold acetone and collected by filtration. The product was dried in an oven at 65° C. overnight under nitrogen sweep to give a light yellow solid; 3.91 g (72.4% of theoretical). The product was identified as N,N-dimethltyrosine N-oxide (DMT-oxide) by NMR analysis. ¹H NMR (D20). δ3.2(m,1H); δ3.45(m,1H); δ3.5(s,3H); δ3.6(s,3H); δ6.9(s,2H); δ7.2(s,2H). Analytical calculated for C11H16NO4: C58.4, H 7.13, N 6.19, O 28.28. Found C58.24, H6.87, N 6.2

### Example 3

### Preparation of pHCA From DMT-Oxide

To a 25 mL 3-neck flask was added 1.0 g (4.419 mmol) N,N-dimethyltyrosine N-oxide (prepared as described in Example 2) and 7.5 g DMAc. A distillation head was attached to the flask and nitrogen sweep was applied such that the nitrogen exited via the distillation head. The reaction mixture was lowered into an oil bath preheated to 130° C. The by-product, N,N-dimethylhydroxylamine, along with some DMAC was collected in the receiving flask. Reaction progress was monitored by sampling and analyzing the samples on HPLC using the method described in General Methods, until all of the starting material was converted, about 30 min. The flask was removed from the oil bath, and the contents of the reaction flask and receiving flask were assayed by HPLC. The reaction pot contained a 75% yield of pHCA. A small amount of p-hydroxystyrene was formed in the receiving flask; 3.3% yield.

### Example 4

### Preparation of P-Hydroxystyrene by Thermal Elimination/Decarboxylation

To a 25 ml 3-neck flask equipped with water circulating distilling head and collecting flask, and under gentle nitrogen sweep, was added 0.52 g (0.0023 mole) of N,N-dimethyltyrosine N-oxide (prepared as described in Example 2) and 5 mL (4.685 g) DMAc. To the resulting solution was added 0.011 g (5 mole %) potassium acetate. The flask was lowered into an oil bath preheated to 123° C. The reaction was sampled and monitored by HPLC. A yield of 96% p-hydroxystyrene (HSM) and 4% pHCA was determined after 2.5 h.

### Example 5

### Optimizing Conditions for Reductive Alkylation of Tyrosine to Give Non Dimethyltyrosine

A Design of Experiment (DOE) analysis was done to determine conditions for maximizing production of N,N dimethyltyrosine. Into a 200 mL stainless steel pressure vessel was added Pd/C as a mol % of tyrosine, as listed in Table 2, followed by nitrogen purge and evacuation cycles. The solvent, either 0.1 M HCl or water as listed in Table 2, was then added followed by adding 2.5 g tyrosine to a final concentration of either 1 M or 0.1 M, and 2.2 equivalents of the 37% formaldehyde solution in water. The vessel was then brought to the desired pressure of hydrogen (see Table 2) at 25° C. and agitated by shaking. Pressure was monitored and the reaction heated to the desired temperature, as listed in Table 2. At the end of the desired reaction time (1 or 8 h) the vessel was purged of hydrogen and flushed with nitrogen. The contents of the vessel were transferred into a glass jar along with one solvent rinse. The combined mixture was analyzed by HPLC using a calibration curve generated with the above described method using authentic N,N-dimethyltyrosine, prepared as described in Example1. Results are tabulated in Table 2.

TABLE 2
Reaction conditions for N,N dimethyltyrosine synthesis, and
yields.

| | | | | | Pd/C | Conc | |
|---|---|---|---|---|---|---|---|
| | Press | temp | Time | | amt | Tyr | Yield |
| Sample | (psi) | (Deg C.) | (h) | Solvent | (mol %) | (M) | DMT |
| 5 | 500 | 25 | 8 | .1M HCl | 1 | 1 | 20.6 |
| 6 | 500 | 25 | 1 | Water | 10 | 1 | 83.3 |
| 7 | 500 | 75 | 8 | Water | 10 | 1 | 84.5 |
| 8 | 500 | 75 | 1 | .1M HCl | 10 | 0.1 | 13.1 |
| 9 | 15 | 25 | 1 | Water | 1 | 0.1 | 13.7 |
| 10 | 15 | 25 | 1 | .1M HCl | 10 | 1 | 37.2 |
| 11 | 15 | 75 | 8 | Water | 10 | 0.1 | 76.0 |
| 12 | 500 | 25 | 8 | Water | 1 | 0.1 | 29.6 |
| 13 | 15 | 75 | 1 | Water | 1 | 1 | 20.9 |
| 14 | 15 | 25 | 8 | .1M HCl | 10 | 0.1 | 56.0 |
| 15 | 500 | 75 | 1 | .1M HCl | 1 | 0.1 | 48.8 |
| 16 | 15 | 75 | 8 | .1M HCl | 1 | 1 | 21.4 |

### Example 6

### Preparation of P-Acetoxystyrene from N,N-Dimethyltyrosine N-Oxide in One Pot

To a 25 ml 3-neck flask equipped with water circulating distilling head and collecting flask, and under gentle nitrogen sweep, was added 0.52 g (0.0023 mole) of N,N-dimethyltyrosine N-oxide (prepared as described in Example 2) and 5 mL (4.685 g) DMAc. To the resulting solution was added 0.011 g (5 mole %) potassium acetate. The flask was lowered into a preheated oil bath at 123° C. The reaction was sampled and monitored by HPLC. A yield of 97% p-hydroxystyrene was determined after 2.4 h. To the solution was added 0.282 g, (2.76 mmol) acetic anhydride and the heating continued for 30 minutes. HPLC analysis using the method described in General Methods with a calibration curve for pAS showed the yield of p-acetoxystyrene to be 86%. The reaction mixture was allowed to cool to room temperature, then poured into 25 ml water. Extraction with ethylacetate, followed by removal of the solvent, gave a yellow oil. Distillation using an oil sublimator at 2 ee-5 torr gave the desired product, as a clear oil.

What is claimed is:

1. A method for the synthesis of p-hydroxycinnamic acid comprising:
a) reacting tyrosine over a metal hydrogenation catalyst with a reaction mixture comprising:
i) at least two equivalents of an aldehyde; and
ii) hydrogen or a hydrogen donor;
to form an N,N-dialkyltyrosine product having the general structure of Formula I: 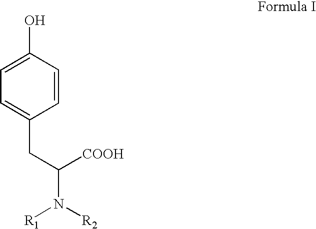
wherein R₁ and R₂ are C1 to C10 linear or branched alkyls;
b) optionally isolating the N,N-dialkyltyrosine product;
c) reacting the N,N dialkyltyrosine product with an oxidizing agent to form N,N dialkyltyrosine N-oxide having the general structure of Formula II: 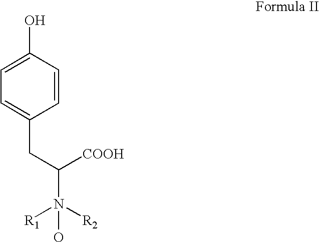
wherein R₁ and R₂ are C1 to C10 linear or branched alkyls;
d) optionally isolating the N,N dialkyltyrosine N-oxide product;
e) heating the N,N dialkyltyrosine N-oxide product to form a mixture of dialkylhydroxylamine by-product and p-hydroxycinnamic acid; and
f) removing dialkylhydroxylamine by-product of step (e) wherein the p-hydroxycinnamic acid product is stabilized.

2. A method for the synthesis of p-hydroxystyrene comprising:
a) reacting tyrosine over a metal hydrogenation catalyst with a reaction mixture comprising:
i) at least two equivalents of an aldehyde; and
ii) hydrogen or a hydrogen donor;
to form an N,N-dialkyltyrosine product having the general structure of Formula I: 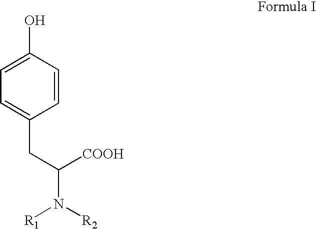
wherein R₁ and R₂ are C1 to C10 linear or branched alkyls;
b) optionally isolating the N,N-dialkyltyrosine product;
c) reacting the N,N dialkyltyrosine product with an oxidizing agent for form an N,N dialkyltyrosine N-oxide product having the structure of Formula II: 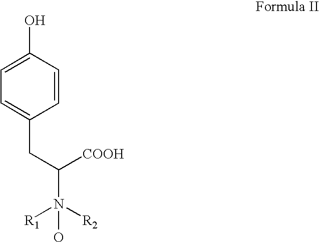
wherein R₁ and R₂ are C1 to C10 linear or branched alkyls;
d) optionally isolating the N,N dialkyltyrosine N-oxide product; and
e) heating the N,N dialkyltyrosine N-oxide product in the presence of a base wherein thermal decomposition occurs to form p-hydroxytsyrene.

3. A method for the synthesis of p-acetoxystyrene comprising:
a) reacting tyrosine over a metal hydrogenation catalyst with a reaction mixture comprising:
i) at least two equivalents of an aldehyde; and
ii) hydrogen or a hydrogen donor;
to form an N,N-dialkyltyrosine product having the general structure of Formula I: 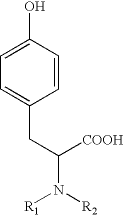
wherein R₁ and R₂ are C1 to C10 linear or branched alkyls;
b) optionally isolating the N,N-dialkyltyrosine product;
c) reacting the N,N dialkyltyrosine product with an oxidizing agentto form an N,N dialkyltyrosine N-oxide product having the general structure of Formula II: 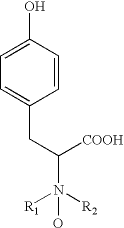
wherein R₁ and R₂ are C1 to C10 linear or branched alkyls;
d) optionally isolating the N,N dialkyltyrosine N-oxide product;
e) heating the N,N dialkyltyrosine N-oxide product with in the presence of base wherein thermal decomposition occurs to produce p-hydroxystyrene;
f) optionally isolating the p-hydroxystyrene of (e); and
g) reacting the p-hydroxystyrene with an acetylating agent wherein p-acetoxystyrene is formed.

4. A method according to any of claims 1, 2, or 3 wherein the metal hydrogenation catalyst of is selected from the group consisting of palladium on carbon, palladium salts on carbon, Raney nickel, platinum, ruthenium, and rhodium.

5. A method according to and of claims 1, 2, or 3 wherein the hydrogen donor of is selected from the group of cylohexene, cyclohexadiene, limonene and ammonium formate.

6. A method according to any of claims 1, 2, or 3 wherein the aldehyde of is selected from the group consisting of: formaldehyde, benzaldehyde, and propionaldehyde

7. A method according to any of claims 1, 2, or 3 wherein the temperature of the reaction of step (a) is from about 20° C. to about 85° C.

8. A method according to any of claims 1, 2, or 3 wherein the oxidizing agent of is selected from the group consisting of meta chloroperbenzoic acid, peroxides and hydroperoxides.

9. A method according to any of claims 1, 2, or 3 wherein the temperature of the reaction of step (e) is from about 60° C. to about 150° C.

10. A method according to either of claims 2 or 3 wherein the base of step (e) is a dialkylhydroxylamine by-product of the thermal decomposition in (e).

11. A method according to either of claims 2 or 3 wherein the base of step (e) comprises a non-amine base.

12. A method according to either of claims 2 or 3 wherein the base is a base catalyst and is provided in catalytic amounts.

13. A method according to claim 11 wherein the process additionally includes removing dialkylhydroxylamine by-product formed during in step (e).

14. A method according to either of claims 2 or 3 wherein the reaction of step (e) optionally comprises an additive selected from the group consisting of: a polymerization inhibitor and a polymerization retarder.

15. The method of claim 14 wherein the polymerization inhibitor is selected from the group consisting of hydroquinone, hydroquinone monomethylether, 4-tert-butyl catechol, phenothiazine, N-oxyl (nitroxide) inhibitors, 4-hydroxy-TEMPO (4-hydroxy-2,2,6,6-tetramethylpiperidin-1-yloxy, CAS#2226-96-2) and Uvinul® 4040 P (1,6-hexamethylene-bis(N-formyl-N-(1-oxyl-2,2,6,6-tetramethylpiperidine-4-yl)amine).

16. The method of claim 14 wherein the polymerization retarder is dinitro-ortho-cresol or dinitrobutyl phenol.

17. The method of claim 3 wherein the acetylating agent is selected from the group consisting of: acetic anhydride and acetyl chloride.
